(19) United States
(12) Patent Application Publication
(10) Pub. No.: US 2014/0088519 A1
(43) Pub. Date: Mar. 27, 2014

(54) Method and Apparatus Providing Interface Between Subcutaneous Port and Patient Tissue

(71) Applicant: Marshall Kerr, Carlsbad, CA (US)

(72) Inventor: Marshall Kerr, Carlsbad, CA (US)

(21) Appl. No.: 13/627,918

(22) Filed: Sep. 26, 2012

(51) Int. Cl. A61M 5/00

(52) U.S. Cl. 604/288.01

(57) ABSTRACT

An apparatus and method for positioning a barrier to cell ingrowth on the exterior surface of a port being implanted under the skin of a patient is provided. The barrier is formed by a film sheet configured to form a cover of the exterior surface of the port and remain mounted thereon in an as-used position. So engaged the film forms a barrier to cell ingrowth upon the port and additionally positions cell growth inhibitors on the exposed surface of the film adjacent to the surrounding cells. Engagement of the film to the port is accomplished by one or a combination of elastic contraction, heat shrinking or adhesive.

This application claims priority to U.S. Provisional Patent Application Ser. No. 61/539,406 filed on Sep. 26, 2011, and incorporated herein in its entirety by reference.

The disclosed device relates to infusion ports. More particularly, it relates to an interface covering for infusion ports which are conventionally implanted subcutaneously in a patient's body. The device and method herein provides an interface between the port exterior surfaces and the patient's surrounding tissue. The film interface helps prevent infections, patient discomfort, and significantly eases the task of removing the port by inhibiting tissue growth upon the port surfaces.

### FIELD OF THE INVENTION

### Background of the Invention

Subcutaneous infusion ports are a preferred form of long term central venous access for patients treated by oncology departments, and for patients requiring long term direct injection of medications. The implanting and use of such ports are preferred, because they provide ease of access for the medical professional for infusion of drugs and are concurrently a less painful and less intrusive manner of infusing those drugs to the patient.

Further, because the port employed to receive and communicate drugs and fluids to the patient is subcutaneous, there is a generally significantly lower infection rate long term versus the employment of multiple or continual incisions to yield required injection sites which are necessary when not using such ports.

Infusion ports have several other advantages over other methods of venous access. One advantage is the ability to implant such ports under local anesthesia as an outpatient procedure, thereby reducing costs to the patient. Another is the long term comfort of the patient who need not endure numerous surgeries to provide required venous access on numerous occasions.

During the implantation process, a surgeon, by forming a skin incision, creates a superficial infusion port pocket under the skin of the patient and above the surrounding flesh or muscle. The port pocket provides a secure position to place the port in a location determined to provide for access to the appropriate blood vessel using a tube or catheter which exits the port. Once the port is implanted, the incision is sutured.

However, such ports can be left implanted for days, weeks, or longer, and such long term implantation of such ports is not without complications. First, the creation of large profile chambers or pockets between the patient's skin and surrounding flesh, over time can cause skin erosions. Such erosions can be complicated by any infection from pathogens which may have accidentally left on the port exterior surface, or from a reaction of the patient's immune system in the tissue surrounding the port caused by its presence.

Additionally, cell growth occurs over time within the pocket for the injection port. Since cells seek attachment to other cells or a support surface this generally causes cells to become attached to the exterior of the implanted port over time. Such a cell ingrowth makes it significantly more difficult to remove the infusion port from the patient. This is because cells in the tissue of the surrounding structures forming the pocket will attach to the exterior of the implanted port due to such cell growth thereon and therebetween.

For the patient this cell growth and attachment generally becomes uncomfortable, especially if accompanied by infection. For the surgeon, it causes the need for a debridement of the cells from the port upon removal of the port from its implant site. Because the debridement must be carefully done to avoid blood vessels, it increases the time for the procedure and the ultimate pain the patient suffers after removal.

Such cell growth is additionally a problem in that the apertures, which are provided on the body of such ports, for the surgeon to suture the port in position under the skin of the patient, must be filled with a material in order to prevent cell growth into the apertures of the port body. Currently, silicone plugs are positioned in the suturing apertures in a timely manufacturing or operative procedure.

Because of the filling of the apertures with a plastic or silicone material which cures to a hardened material, surgeons must, during implantation of a port, use significantly more hand and finger pressure and strength to generate it. This is required to force the needle and suture through the cured material positioned within the apertures in the port while suturing the port into position in an implantation pocket.

As such, there exists a continual unmet need for a means for a medical professional to retard or prevent cell ingrowth and overgrowth upon the exterior surfaces of implanted infusion ports and the like. Such a device should be easily engaged to the exterior surface of the implant in a sterile environment. Such a device should prevent, or significantly impair the ingrowth or overgrowth of cells upon the port exterior surface during the duration of implantation in the patient to aid in an easy removal. Finally, such a device should eliminate the need to fill suturing holes or apertures in the port body, with cured plastic material, which subsequently increases the surgeon's effort and time during implantation and suturing therethrough.

With respect to the above, before explaining at least one preferred embodiment of the invention in detail or in general, it is to be understood that the invention is not limited in its application to the details of construction and to the arrangement of the components or the steps set forth in the following description or illustrated in the drawings. The various apparatus and methods of the invention are capable of other embodiments, and of being practiced and carried out in various ways, all of which will be obvious to those skilled in the art once the information herein is reviewed. Also, it is to be understood that the phraseology and terminology employed herein are for the purpose of description and should not be regarded as limiting.

As a consequence, those skilled in the art will appreciate that the conception upon which this disclosure is based may readily be utilized as a basis for designing and producing interface covers to fit on infusion and other implantable ports used for long term delivery of drugs and medicine to patients and the like, and for carrying out the several purposes of the present disclosed device and method. It is important, therefore, that the embodiments, objects and claims herein, be regarded as including such equivalent construction and methodology insofar as they do not depart from the spirit and scope of the present invention.

### SUMMARY OF THE INVENTION

The interface cover device herein is configured for employment engaged over and in combination with an injection port (or portacath) which is a small medical appliance that is conventionally installed in a formed pocket beneath the skin of a patient. Such ports are designed for implantation under the skin of a patient using an incision which is then sutured.

Such infusion ports conventionally have a septum or membrane cover above an interior reservoir which is positioned on an upper surface of the port which in this case is closest to the patient's skin. This septum provides a self-sealing means to communicate with an underlying reservoir. It is adapted to be pierced multiple times by a needle or other means to communicate fluid, medicine or drugs into the underlying reservoir or for the taking of blood samples therefrom on numerous occasions.

A catheter, or other means for sealed communication of a lumen between a blood vessel, and the interior reservoir connects the reservoir under the septum to an internal blood vessel such as a vein or artery.

It is the exterior surface of the body of the port, and also the septum, which will suffer from cell ingrowth during the duration of the implantation. Further, it is this same surface that can carry pathogens into the body of the patient if improperly sterilized or contaminated somehow prior to implant.

The interface cover device herein, allowing the method herein, is formed of a thin film. The film sheet is configured to engage over such an implantable port and to cover a majority of its external surface area in an as-used position or engagement. Thus, in the as-used position, with the film sheet engaged upon and covering such a port, the device provides a separation or barrier between surrounding tissue and the surface of the port and a means to prevent cell ingrowth and overgrowth on the implanted port and its surfaces.

The film sheet may be custom cut and dimensioned to fold and attach to the exterior surface of the port. In the simplest form of the device, the film sheet is folded around the entire port and adhered using adhesive or a shrinking by heat and resulting compression, or similar means for adhesion. In other preferred modes, the film sheet is enhanced using multiple materials such as a heat-shrinking polymeric material which contacts the port surface on a first side and has a cell growth inhibiting material such as a silicone or titanium layer or coating or surface on the opposite or second side.

The entire film sheet, or the exterior facing surface, is formed as noted of a material that inhibits cell growth and/or cell attachment to the underlying port and its surfaces. The inhibiting material can be one or a combination of materials from a group including silicone, nano silicone, titanium, PTFE, silver ions, or other flexible surface materials which will cause cell growth limiting substances in addition to the action of the silicone film forming the device, and/or infused with medicines to prevent infection such as antibiotics which would be delivered slowly and directly into the pocket formed for the implant where infection is a frequent problem.

Engaged over the body of such a port, the device provides a cover over the open apertures employed for suturing the port in place in the pocket formed in the tissue under a patient's skin. This cover allows for the suture apertures to be left open rather than filled. Thereby making it easier for the surgeon to suture through the open ports to mount the port in place.

The device would be formed in a sheet which is configured to form fit the contours of the implanted port and may be formed to cover all but the septum in one mode, or as a donut shape in another which would leave the septum and/or lower surface exposed. Additionally, the film sheet may be formed of heat-shrink material at the first surface with a silicone layer or surfacing on the opposite surface, using a material such as Olefin polymers with a layer of silicone such as the film CLYSAR HPS which is manufactured by the Dupont company. Employing a film sheet with the silicone exterior or second surface, the port may be wrapped in the film and exposed to heat which will shrink it to the conforms of the port which the sheet maintains once the heat is removed. This is a particularly preferred mode of the device and method herein since it provides the most contoured fit of the film to the port thereby forming a silicone barrier between the port surface and the tissue of the formed pocket it occupies.

If provided with heat-shrink film with a silicone or other exterior surface, the sheets will shrink to fit so long as they are reasonably formed to the correct size. With the addition of an adhesive to the first surface contacting the port, adhesion is enhanced also. The film sheets may be provided in sterile envelopes or from containers of dispensable sheets, which are maintained sterile until employed and are configured to engage over the port and shrink to fit and adhere to it.

The foregoing has outlined rather broadly the more pertinent and important features of the device and method herein employing a film formed to cover and interface between an infusion port or other implanted device and the patient's cells in the pocket surrounding it on an implant in order that the detailed description of the invention that follows may be better understood so that the present contribution to the art may be more fully appreciated. Additional features of the invention may be described hereinafter which form the subject of the claims of the invention. It should be appreciated by those skilled in the art that the conception and the disclosed specific embodiments may be readily utilized as a basis for modifying or designing other interface coverings for implanted ports for carrying out the same purposes of the present invention. It should also be realized by those skilled in the art that such equivalent constructions and methods do not depart from the spirit and scope of the invention as set forth herein and are considered within the scope of this invention.

Further, in this respect, before explaining at least one embodiment of the invention in detail, it is to be understood that the invention is not limited in its application to the details of construction and to the arrangement of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

### THE OBJECTS OF THE INVENTION

It is therefore an object of the present invention to provide a cover and interface between the exterior surfaces of an implanted infusion port or other implanted device and surrounding tissue during the term of implantation.

It is another object of this invention to provide such a device and method that may be easily incorporated for engagement over existing implantable ports and be easily interfaced with the installed base of medical equipment at medical facilities through the use of film sheets which are configured to conform, and/or shrink to conform to the exterior contours and surfaces of the implanted port.

The foregoing has outlined some of the more pertinent objects of the invention. These objects should be construed to be merely illustrative of some of the more prominent features and applications of the intended invention. Many other beneficial results can be attained by applying the disclosed method and port interface device in a different manner or by modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### BRIEF DESCRIPTION OF THE DRAWINGS

The accompanying drawings, which are incorporated herein and form a part of this specification, illustrate embodiments of the invention and together with the detailed description, serve to explain the principles of this invention.

FIG. 1 depicts a perspective view of a conventional infusion port device employable with the film interface herein.

FIG. 2 depicts a top view of the device of FIG. 1, showing the septum surface of the implantable port and surrounding top surface.

FIG. 3 depicts a side view thereof.

FIG. 4 depicts a bottom view of a port such as in FIG. 1.

FIG. 5 depicts a side view of the device herein showing the film sheet of material defining the cover formed to engage over the conventional port of FIGS. 1-4.

FIG. 6 depicts a view of the device herein showing the film sheet engaged on a port of FIG. 3.

FIG. 7 depicts a top view of a conventional port as in FIG. 2, with the device engaged to cover the port surfaces surrounding the septum.

FIG. 8 depicts a mode the device wherein the film sheet is configured for a wrapping and covering of the entire surface of the port.

FIG. 9 depicts a second mode of the bottom of the interface device herein, having an elastic center aperture employable for engaging the flexible film material over the port it surrounds.

FIG. 10 depicts a container for holding and dispensing the properly configured film sheets during implantation.

FIG. 11 depicts an envelope or package employed to maintain the film sheet material within, sterile until engaged to the port.

FIG. 12 depicts the film sheet having an optional aperture for surrounding the septum and having a first side surface for contact and adherence to the underlying port, and a second surface layered or coated or infused with a cell growth retardant and/or pathogen growth inhibitant.

FIG. 13 depicts a the film sheet from FIG. 12 formed to a three-dimensional oversized covering in a shape configured to engage over the contours of the body of the port after insertion and using a heat source to shrink the film, with optionally adhesive on the first surface.

FIG. 14 shows the film sheet adhered and contoured to the bottom surface of the infusion port.

### DETAILED DESCRIPTION OF THE PREFERRED EMBODIMENTS

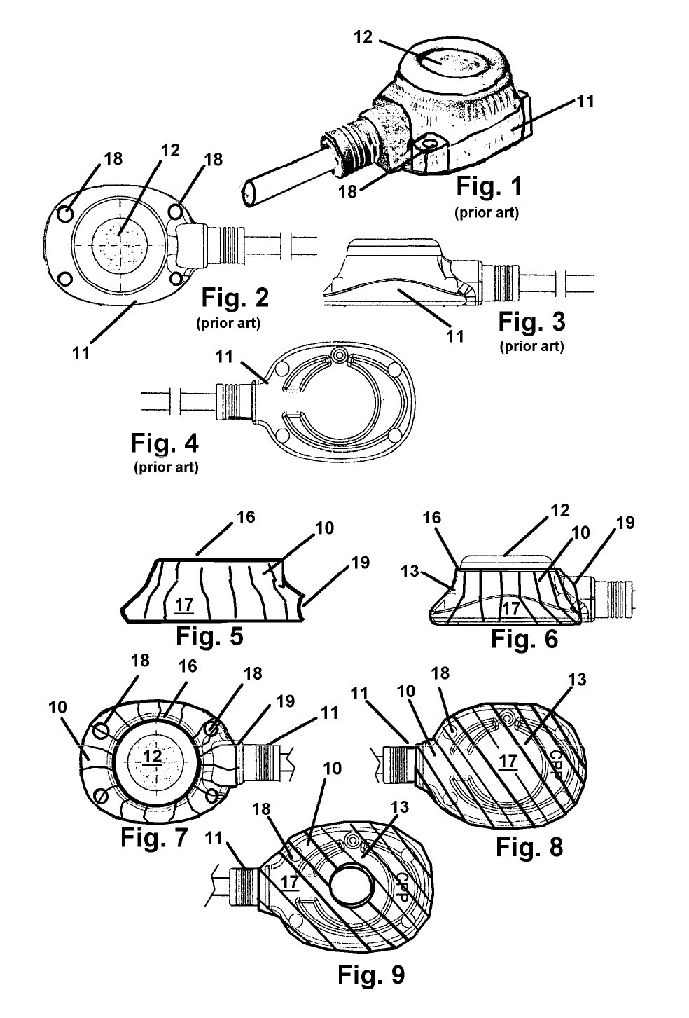

Referring now to the drawings 1-14, wherein similar parts of the invention are identified by like reference numerals, there is shown various views of a conventional prior art pressure port 11 in FIGS. 1-4. Such ports are conventionally implanted, as noted above, under the skin of a patient in pockets formed between overlying skin and surrounding tissues.

The device 10 and method herein employs a flexible film 13 sheet as shown in FIG. 12 which has a first side surface 15 and an opposite second side surface 17 formed as a single layer or laminate. The dimensions of the film 13 sheet are configured to fully cover the desired exterior surfaces of a conventional port 11 as in FIGS. 1-5. If the material forming the film 13 is heat-shrinkable material, which will shrink and conform to the port exterior surfaces when heated, then the dimensions of the sheet of film 13 need to accommodate the amount of shrinkage and fully cover all or the intended exterior surface of the port 11. A means for imparting heat to shrink the film 13 sheet once engaged to the port 11 could be a blow-dryer however a UV Light source would be more preferred due to the fact it does not disrupt air in the sterile field of the procedure, and, UV light will kill most pathogens should they have somehow gotten on the surface of the port 11 or the film 13.

As shown in FIG. 5 which shows the device 10 formed by the method herein, from a side view, the formed device 10 employs the film 13 sheet. The entire sheet of film 13 may be formed as a single film layer of material which inhibits cell growth and/or cell attachment to the underlying port 11 and its surfaces, or the second surface 17 may be have such cell growth inhibiting material splattered, sprayed, coated, laminated or otherwise placed upon and covering substantially the entire second surface 17. Such cell growth inhibiting material can include one or a combination of materials, from a group of cell growth inhibiting materials, including silicone, nano silicone, titanium, PTFE, and silver ions. Currently one or a combination of silicone or nano silicone is particularly preferred for forming, or covering the second surface 17 as experimentation has shown silicone is inert as to causing cell irritation and is an exceptional inhibitant of cell growth. Titanium is also an excellent material for the same reasons and because it enhances the lubricity of the area surrounding the implant.

The film 13, either planar or formed as a three-dimensional covering, has the second surface 17 which will contact surrounding tissue when the port 11 is implanted, and provide a barrier between the cells and tissue and any adhesion to the exterior surface of the port 11. Of course the second surface 17 of the film can be made of, coated with, laminated with, deposited with, or impregnated with, other materials which inhibit cell growth would occur to those skilled in the art, and such is anticipated within the scope of this invention.

In FIG. 6 is shown the assembled device 10 with the film 13 such as shown in FIG. 12 or 13, is engaged and conformed to the installed as-used position covering the port 11. If the optional aperture 16 is provided the film 13 can be engaged to leave the septum 12 uncovered to provide uninhibited access for injections. If the aperture 16 is to be employed, it would be formed in either the planar sheet 13 of FIG. 12 or the contoured sheet 13 of FIG. 13 in a size to cover the port 11 surface surrounding the septum 12 when the film 13 is operatively engaged in the as-used position.

To that end, the device 10 can either use film 13 which is either molded or cut or thermoformed, or otherwise formed to such configuration, with an aperture 16 sized to engage around the septum 12 and to allow needle access to the septum 12. If a heat shrink material is not employed, in a similar fashion an elastic surrounded or formed aperture 16 can be formed to stretch over the perimeter of the port 11, and allow for installation of the film 13 sheet on the port 11 much like a sock is engaged to a foot. This elastic mode of the film 13 might be combined with a means for imparting heat to the film such as a UV lamp, and provide for a shrinking of the film 13 to a tight fit and adhesive or other contact of the first surface 15 with the exterior surface of the port 11 surrounding the septum 12.

FIG. 7 shows the assembled device 10 with the film 13 installed to cover the body of the port 11, and with the aperture 16 in a registered engagement to allow access to the septum 12. Also shown is the film 13 covering the suture apertures 18 protecting them from cell ingrowth, while allowing the elimination of the hard plugs therein which conventionally inhibit the surgeon's suturing.

FIG. 8 depicts the assembled device 10 with the film 13 having its first surface 15 engaged and covering the entire bottom exterior surface of the port 11. Mounting of the film 13 to the port 11 is accomplished, as noted, using a means for heating and heat-shrink film 13 which when wrapped around the port 11 from the planar sheet of FIG. 12, or engaged using the dimensioned sheet of FIG. 13, will shrink and adhere to the exterior of the port 11. Adherence to the exterior surface of the port 11 may be with one or a combination of an adhesive or attachment of the film 13 itself.

Alternatively, if the film 13 is configured in the dimensioned shape such as that of FIG. 13, which is slightly larger than the port 11 but in the same general shape, the threaded end 33 of the port can be inserted through the septum aperture 16, and exited through a side aperture 19 and the configured film 13, forming the device 10, will contact the exterior surfaces of the port 11 when heated to shrink, and adhere thereto using adhesive positioned on the first surface 15 or just by contact with the first surface 15. Or if the film 13 is elastic in nature, a sliding of the port 11 and threading through the two apertures in the above referenced manner will allow the film 13 to then retract and engage upon the exterior of the port 11.

FIG. 9 depicts a second mode of the bottom of the assembled device 10 herein, having a center aperture 16 employable for engaging the port 11 with the film 13 of the assembled device 10. This formation would place a small opening where the aperture 16 is positioned, in another mode of installation.

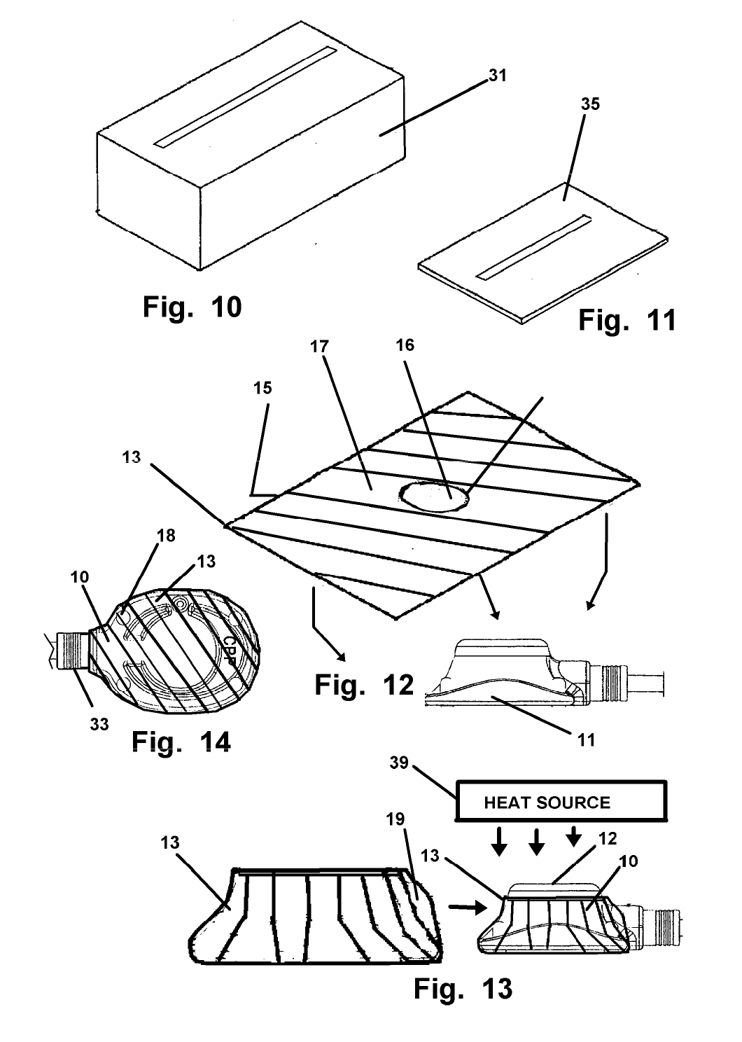

FIG. 10 depicts a container 31 for holding and dispensing the properly configured film 13 during implantation. If in a planar sheet 13 such as in FIG. 12, it may be dispensed directly, or using a removable cover 35. Or as shown in FIG. 11 depicts an envelope or removable cover 35 can be employed and packed with the port 11 kit, to maintain the sterility of the film 13 until engaged on the port 11. The film 13 can be positioned in the cover 35 or container 31 in either the cut planar sheet mode of the film 13 of FIG. 12, or the contoured sheet mode of the film 13 of FIG. 13.

Once removed from the container 31 and or cover 35, the film 13 is engaged upon the port 11 in a manner consistent with the mode of provision of the sheet 13. If planar as in FIG. 12 the film 13 is wrapped around the port 11 as in FIG. 12 and adhered thereto using adhesive or the attraction of the first side surface 15 to the port surface. If the film is provided in a dimensioned form as in FIG. 13, using either elastic film 13 which will contract and engage the port 11, or as a heat shrinking film which is exposed to a UV source or other means to heat the film 13, it too can be provided in a container 31 and/or the cover 35 to maintain sterility till engaged to the port 11.

FIG. 12 as noted depicts the film 13 in a sheet having an optional aperture for surrounding the septum 12 and having a first side surface 15 for contact and adherence to the underlying port 11. Also shown is the second surface 17 layered or coated or infused with a cell growth retardant and/or pathogen growth inhibitant noted herein.

FIG. 13 depicts the film 13 sheet from FIG. 12 formed in the three dimensional configuration, which substantially follows to the shape of the exterior surface of the port 11. As noted it may be elastic film 13, or as depicted, formed oversized and then reduced in size using a heat source 39 such as a UV light or hot air, to cover the exterior contours of the exterior surface of the port 11. Engagement with the surface can be as noted one or a combination of adhesion means such as compression in a heat shrink process and/or adhesive, and or an ionic attraction of the first surface 15 to the surface of the port 11.

FIG. 14 shows the film 13 sheet from FIG. 12 or 13, adhered against and the bottom surface of the infusion port 11 and continuing up the contours of the sides of the port 11.

As such, the system and device 10 provided by the engagement of the film 13 sheet to cover the all or substantially all of the exterior surface of an implanted port 11, provides great utility to the surgeon and patient by decreasing cell ingrowth which is hard to remove and decreasing irritation and infection while implanted. Using the film 13 which is sized to envelop all or most of the exterior surface of the port 11 and adhere thereto on a first side surface of the film, the film 13 in this as-used position provides a barrier to cell ingrowth to the exterior of the port 1.

Means for engagement may be my forming the sheet 13 similar in configuration but larger than the exterior of the port 11 and using a heat source to shrink the film, or by using an elastic film sized equal to or slightly larger than the area of the exterior of the port 11, and stretching the film 11 thereover. Or, the film 13 can be provided as a pre-cut planar sheet which may be wrapped to envelop most of the exterior surface of the port 11. Or, as noted the film 13 may be formed in a three dimensional configuration larger than the exterior of the port 11 and then shrunk to a compressive and/or adhesive engagement of the first surface 15 with the exterior of the port 11.

In this as-used configuration with the first surface 15 of the film 13 in a contact and engaging the exterior of the port 11, the primary object of presenting a second surface 17 of the film 13 provides for contact with surrounding cells as a barrier to cell ingrowth is accomplished. Further in all modes the second surface 17 can be a laminate or spray coated, or sputter coated, surface of one or a combination of cell ingrowth inhibitors such as silicone and titanium or any of the aforementioned cell growth inhibitors.

While all of the fundamental characteristics and features of the disclosed port interface device and method herein have been described herein, with reference to particular embodiments thereof, a latitude of modification, various changes and substitutions are intended in the foregoing disclosure and it will be apparent that in some instance, some features of the invention will be employed without a corresponding use of other features without departing from the scope of the invention as set forth. It should be understood that such substitutions, modifications, and variations may be made by those skilled in the art without departing from the spirit or scope of the invention. Consequently, all such modifications and variations are included within the scope of the invention as defined herein.

1. An apparatus providing a barrier to cell ingrowth on the exterior surface of a port implanted under the skin of a patient, comprising:
a film sheet having a first surface and a second surface opposite said first surface;
said film sheet configured to form a cover of said exterior surface of said port, after said first surface is positioned thereon, thereby positioning said film sheet in an as-used position;
means to maintain said first surface adjacent to said exterior surface and thereby maintain said film sheet in said as-used position;
said second surface of said film sheet having a cell growth inhibitor thereon;
said cell growth inhibitor inhibiting the growth of cells of said patient and defining a barrier for attachment of said cells to said surface of said port; and
whereby said film sheet can be engaged to said as-used position on a said port during an implantation thereof under said skin of said patient.

2. The apparatus providing a barrier to cell ingrowth of claim 1 wherein said means to maintain said first surface adjacent to said exterior surface and thereby maintain said film sheet in said as-used position, comprises:
adhesive on said first surface.

3. The apparatus providing a barrier to cell ingrowth of claim 1 wherein said means to maintain said first surface adjacent to said exterior surface and thereby maintain said film sheet in said as-used position, comprises:
said film sheet formed to a shape conforming to said exterior surface of said port wherein said first surface has a configuration of a cavity, similar to that of said exterior surface of said port;
said film sheet formed of elastic material;
an aperture formed in said film sheet sized to allow insertion of said port therethrough, and a stretching of said film from a relaxed size, to an enlarged sized, during said insertion of said port through said aperture and into said cavity;
a return of said film to said relaxed sized providing a compressive frictional engagement of said first surface to said exterior surface of said port.

4. The apparatus providing a barrier to cell ingrowth of claim 1 wherein said means to maintain said first surface adjacent to said exterior surface and thereby maintain said film sheet in said as-used position, comprises:
said film sheet formed to a shape conforming to said exterior surface of said port wherein said first surface has a configuration of a cavity;
said cavity being larger than the area of said exterior surface of said port;
said film being sheet heat shrink material;
an aperture formed in said film sheet sized to allow insertion of said port therethrough, during said insertion of said port through said aperture and into said cavity; and
said film shrinking upon communication of a heat source thereto, to form a compressive frictional engagement of said first surface to said exterior surface of said port.

5. The apparatus providing a barrier to cell ingrowth of claim 2 wherein said means to maintain said first surface adjacent to said exterior surface and thereby maintain said film sheet in said as-used position, additionally comprises:
said film sheet formed to a shape conforming to said exterior surface of said port wherein said first surface has a configuration of a cavity, similar to that of said exterior surface of said port;
said film sheet formed of elastic material;
an aperture formed in said film sheet sized to allow insertion of said port therethrough, and a stretching of said film from a relaxed size, to an enlarged sized, during said insertion of said port through said aperture and into said cavity;
a return of said film to said relaxed sized providing a compressive adhesive engagement of said first surface to said exterior surface of said port.

6. The apparatus providing a barrier to cell ingrowth of claim 2 wherein said means to maintain said first surface adjacent to said exterior surface and thereby maintain said film sheet in said as-used position, additionally comprises:
said film sheet formed to a shape conforming to said exterior surface of said port wherein said first surface has a configuration of a cavity;
said cavity being larger than the area of said exterior surface of said port;
said film being sheet heat shrink material;
an aperture formed in said film sheet sized to allow insertion of said port therethrough, during said insertion of said port through said aperture and into said cavity; and
said film shrinking upon communication of a heat source thereto, to form a compressive adhesive engagement of said first surface to said exterior surface of said port.

7. The apparatus providing a barrier to cell ingrowth of claim 1 wherein said cell growth inhibitor comprises one or a combination of cell growth inhibiting materials from a group of inhibiting materials including silicone, nano silicone, titanium, PTFE, and silver ions.

8. The apparatus providing a barrier to cell ingrowth of claim 2 wherein said cell growth inhibitor comprises one or a combination of cell growth inhibiting materials from a group of inhibiting materials including silicone, nano silicone, titanium, PTFE, and silver ions.

9. The apparatus providing a barrier to cell ingrowth of claim 3 wherein said cell growth inhibitor comprises one or a combination of cell growth inhibiting materials from a group of inhibiting materials including silicone, nano silicone, titanium, PTFE, and silver ions.

10. The apparatus providing a barrier to cell ingrowth of claim 4 wherein said cell growth inhibitor comprises one or a combination of cell growth inhibiting materials from a group of inhibiting materials including silicone, nano silicone, titanium, PTFE, and silver ions.

11. The apparatus providing a barrier to cell ingrowth of claim 5 wherein said cell growth inhibitor comprises one or a combination of cell growth inhibiting materials from a group of inhibiting materials including silicone, nano silicone, titanium, PTFE, and silver ions.

12. The apparatus providing a barrier to cell ingrowth of claim 6 wherein said cell growth inhibitor comprises one or a combination of cell growth inhibiting materials from a group of inhibiting materials including silicone, nano silicone, titanium, PTFE, and silver ions.

13. A method of forming a barrier to cell growth on a port being surgically positioned under the skin of a patient comprising employing the apparatus of claim 1, comprising the steps of:
positioning a film sheet having a first surface and a second surface opposite said first surface to form a cover of said exterior surface of said port by placing said first surface in contact with said exterior surface of said port;
employing means to maintain said first surface adjacent to said exterior surface of said port, to maintain said film in an as-used position; and
thereby positioning said second surface of said film sheet having a cell growth inhibitor thereon adjacent to cells surrounding said port.
